(19) United States
(12) Patent Application Publication
(10) Pub. No.: US 2022/0359042 A1
(43) Pub. Date: Nov. 10, 2022

(54) DATA SIFTING METHOD AND APPARATUS

(71) Applicant: DELTA ELECTRONICS, INC., Taoyuan City (TW)

(72) Inventors: Ming-Che HSIEH, Taoyuan City (TW); Jie-Sheng YANG, Taoyuan City (TW)

(21) Appl. No.: 17/449,952

(22) Filed: Oct. 5, 2021

(30) Foreign Application Priority Data
May 7, 2021 (CN) 202110495855.6

(51) Int. Cl. G16B 40/10; G16B 25/20; C12Q 1/68

(52) U.S. Cl. CPC G16B 40/10; G16B 25/20; C12Q 1/68

(57) ABSTRACT

A data sifting method applied to a growth type curve including a plurality of data points, and includes: calculating a plurality of first derivative values corresponding to the data points; searching at least one local maximum value from the first derivative values; determining whether a part of the first derivative values adjacent to the at least one local maximum value are all positive; determining one of the first derivative values after a predetermined effective cycle number or the at least one local maximum value as a target maximum value according to a determination result; deriving a basic cycle number according to a target cycle number corresponding to the target maximum value; and setting a baseline of the growth type curve according to the basic cycle number to calculate a first Cq value according to the adjusted growth type curve. The present disclosure further provides a data sifting apparatus.

### CROSS-REFERENCE TO RELATED APPLICATION

This application claims priority to China Application Serial Number 202110495855.6, filed on May 7, 2021, which is herein incorporated by reference in its entirety.

### BACKGROUND

### Field of Invention

This disclosure relates to a data pre-screening method and apparatus, and in particular to a data pre-screening method and apparatus applied to a growth type curve of a polymerase chain reaction (PCR).

### Description of Related Art

During the nucleic acid testing, the fluorescent signals generated at each cycle number of the polymerase chain reaction process would be detected generally. A curve plotted based on the fluorescent signals generated at each cycle number is known as a growth type curve. Conventionally, a Cq (cycle of quantification) value (which represents the cycle number at which the fluorescent signal crosses a predetermined threshold value) is often calculated through the growth type curve, to interpret the test result (i.e., positive test result or negative test result). However, the instability of the fluorescent signals often affects the calculation of Cq value, to further affect the interpretation of the nucleic acid testing.

### SUMMARY

An aspect of present disclosure relates to a data pre-screening method or a data sifting method applied to a processor. The processor is coupled to a memory storing a growth type curve, the growth type curve is generated by performing a polymerase chain reaction on an object under test, the growth type curve comprises a plurality of data points, and the data sifting method includes: calculating a plurality of first derivative values corresponding to the data points; searching at least one local maximum value from the first derivative values; determining whether the at least one local maximum value is greater than a first threshold, and determining whether a part of the first derivative values adjacent to the at least one local maximum value are all positive; determining one of the first derivative values after a predetermined effective cycle number or the at least one local maximum value as a target maximum value according to a determination result; obtaining a target cycle number corresponding to the target maximum value; deriving a basic cycle number according to the first derivative value corresponding to the target cycle number; adjusting the data points according to the basic cycle number to form a baseline of the growth type curve; and calculating a first Cq value according to the adjusted growth type curve.

Another aspect of present disclosure relates to a data sifting apparatus. The data sifting apparatus includes a memory and a processor. The memory is configured to store a growth type curve including a plurality of data points, wherein the growth type curve is generated by performing a polymerase chain reaction on an object under test. The processor is coupled to the memory and is configured to execute following operations: calculating a plurality of first derivative values corresponding to the data points; searching at least one local maximum value from the first derivative values; determining whether the at least one local maximum value is greater than a first threshold, and determining whether a part of the first derivative values adjacent to the at least one local maximum value are all positive; determining one of the first derivative values after a predetermined effective cycle number or the at least one local maximum value as a target maximum value according to a determination result; obtaining a target cycle number corresponding to the target maximum value; deriving a basic cycle number according to the first derivative value corresponding to the target cycle number; adjusting the data points according to the basic cycle number to form a baseline of the growth type curve; and calculating a first Cq value according to the adjusted growth type curve.

### BRIEF DESCRIPTION OF THE DRAWINGS

FIG. 1 is a block diagram of the data sifting apparatus in accordance with some embodiments of the present disclosure;

FIGS. 2A-2B are flow diagrams of the data sifting method in accordance with some embodiments of the present disclosure;

FIG. 3 is a schematic diagram of the typical growth type curve, the first derivative curve and the second derivative curve in accordance with some embodiments of the present disclosure;

FIG. 4 is a schematic diagram of the atypical growth type curve, the first derivative curve and the second derivative curve in accordance with some embodiments of the present disclosure;

FIG. 5 is a schematic diagram of another atypical growth type curve in accordance with some embodiments of the present disclosure; and

FIG. 6 is a schematic diagram of yet another atypical growth type curve in accordance with some embodiments of the present disclosure.

### DETAILED DESCRIPTION

The embodiments are described in detail below with reference to the appended drawings to better understand the aspects of the present disclosure. However, the provided embodiments are not intended to limit the scope of the disclosure, and the description of the structural operation is not intended to limit the order in which they are performed. Any device that has been recombined by components and produces an equivalent function is within the scope covered by the disclosure.

The terms used in the entire specification and the scope of the patent application, unless otherwise specified, generally have the ordinary meaning of each term used in the field, the content disclosed herein, and the particular content.

The terms “coupled” or “connected” as used herein may mean that two or more elements are directly in physical or electrical contact, or are indirectly in physical or electrical contact with each other. It can also mean that two or more elements interact with each other.

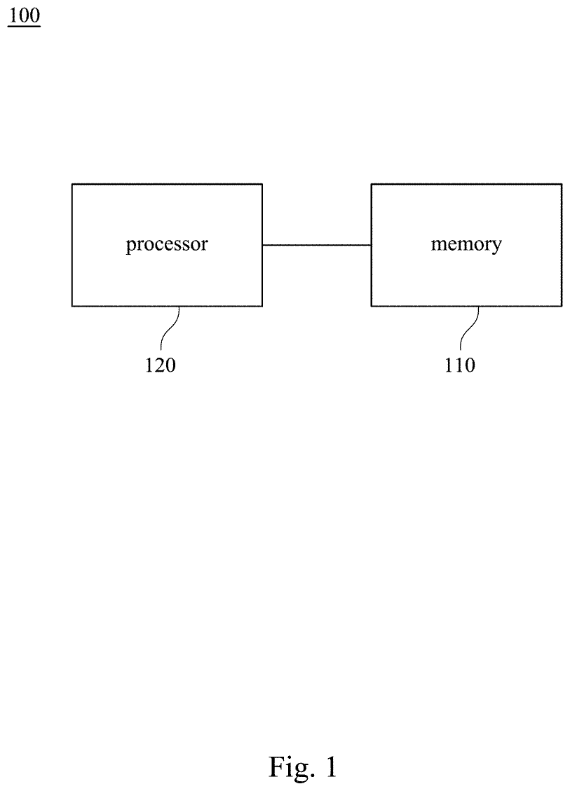

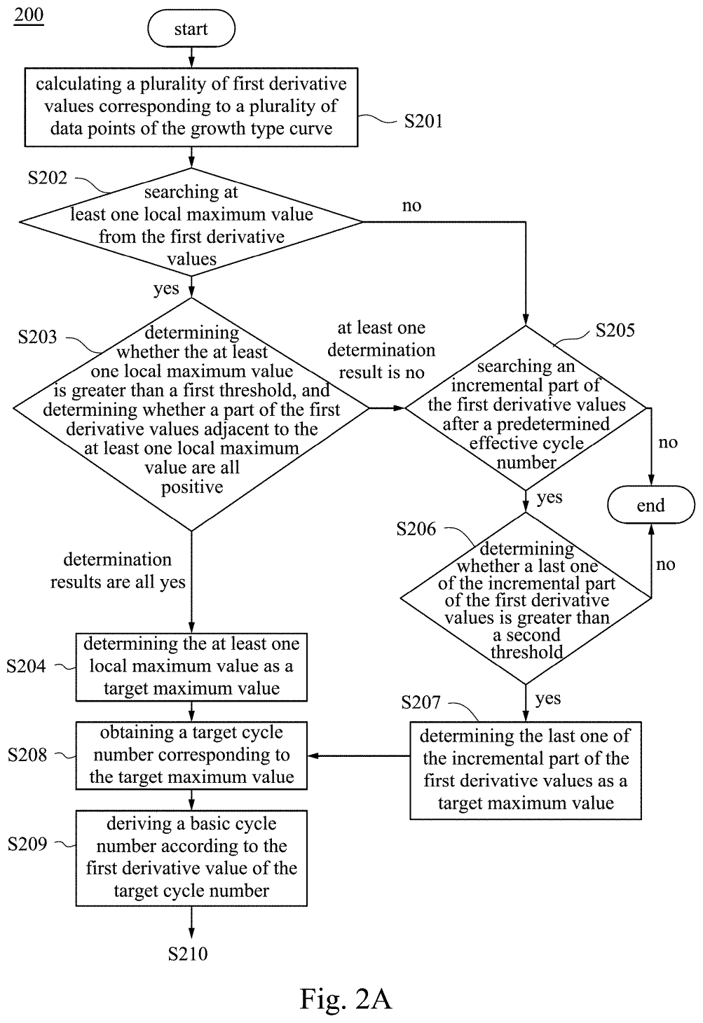

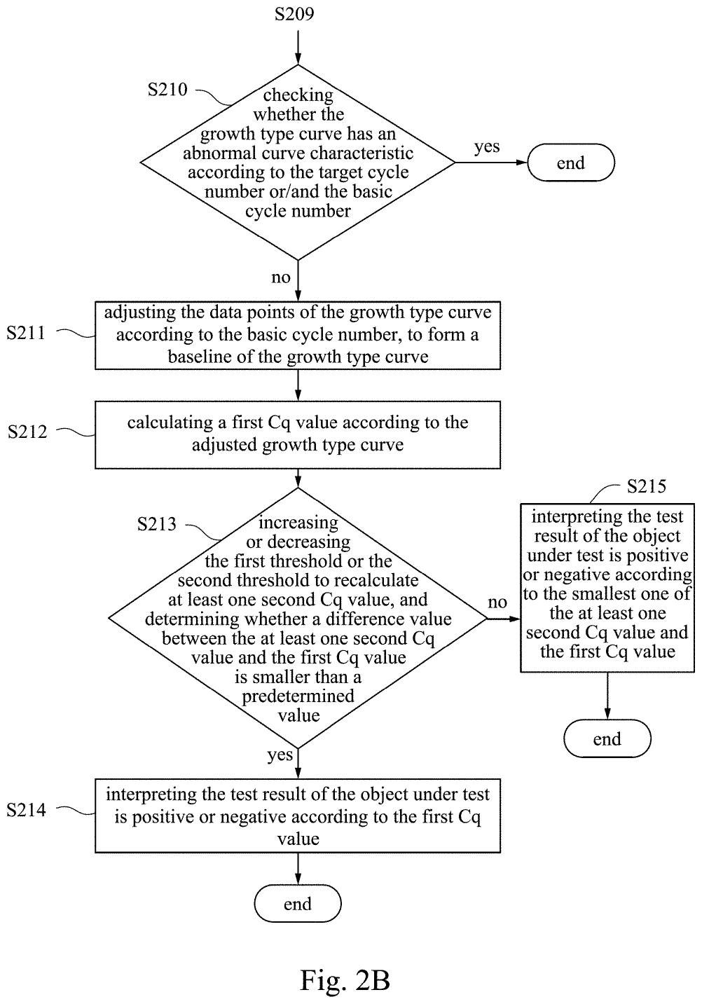

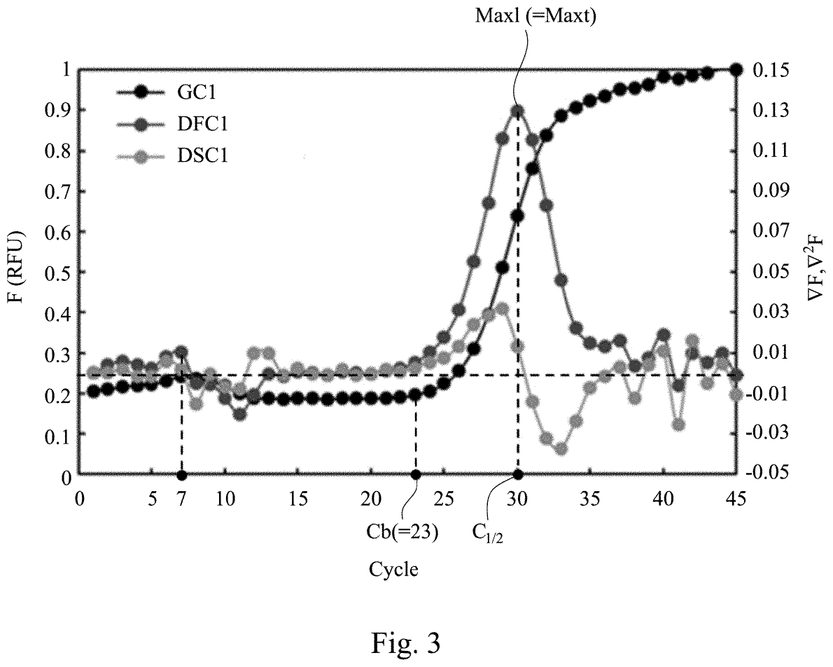

Referring to FIG. 1, FIG. 1 depicts a data sifting apparatus 100 in accordance with some embodiments of the present disclosure. The data sifting apparatus 100 includes a memory 110 and a processor 120. The data sifting apparatus 100 is configured to perform a data sifting operation (e.g., a data sifting method 200 as shown in FIGS. 2A-2B) on growth type curves (e.g., GC1, GC2, GC3 and GC4 as shown in FIGS. 3, 4, 5 and 6), so as to determine whether to further analyze the growth type curve. For example, a Cq (cycle of quantification) value for interpreting the result of the nucleic acid testing is calculated according to the growth type curve. In some embodiments, the growth type curve is generated by performing a polymerase chain reaction (PCR) on an object under test.

As shown in FIG. 1, the processor 120 is coupled to the memory 110. In some embodiments, the memory 110 can be implemented by a non-transitory computer readable storage medium (e.g. random access memory (RAM), read only memory (ROM), hard disk drive (HDD), solid-state drive (SSD)). The processor 120 can be implemented by one or more central processing unit (CPU), application-specific integrated circuit (ASIC), microprocessor, system on a Chip (SoC) or other suitable processing units.

Referring to FIGS. 2A-2B, FIGS. 2A-2B depict a data sifting method 200 in accordance with some embodiments of the present disclosure. The data sifting method 200 can be executed by the processor 120 of the FIG. 1. However, the present disclosure is not limited herein. As shown in FIGS. 2A-2B, the data sifting method 200 includes operations S201-S215.

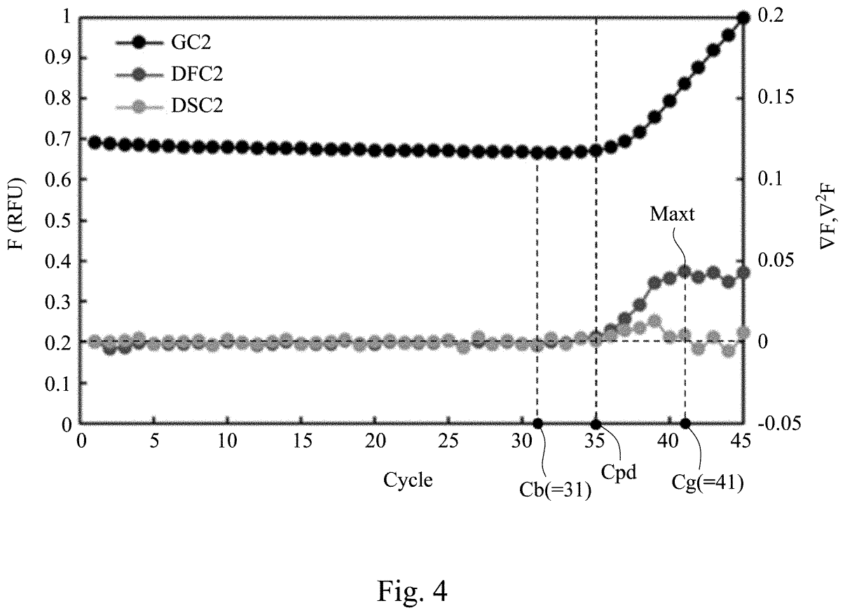

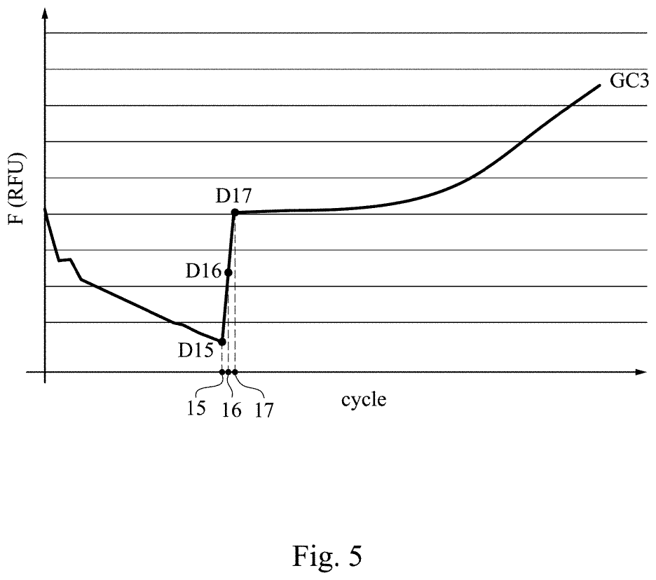

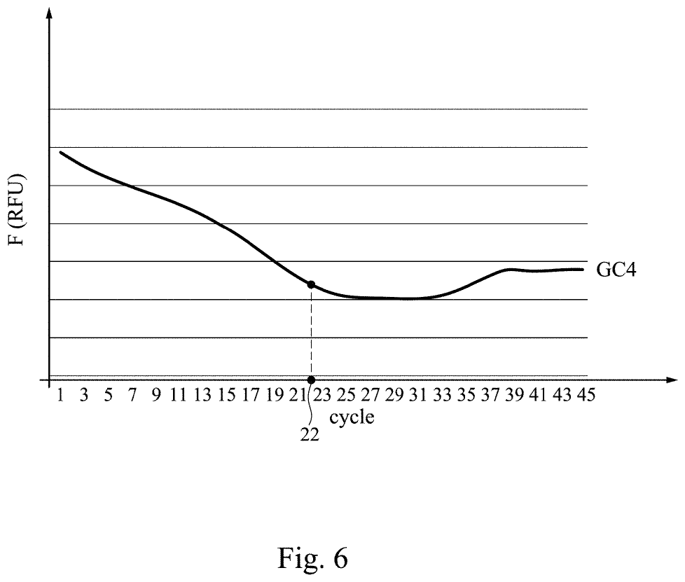

For better understanding the present application, the data sifting method 200 would be described in detail below with reference to FIGS. 3, 4, 5 and 6. FIG. 3 is a schematic diagram of a typical growth type curve GC1, a first derivative curve DFC1 and a second derivative curve DSC1 in accordance with some embodiments of the present disclosure. FIG. 4 is a schematic diagram of an atypical growth type curve GC2, a first derivative curve DFC2 and a second derivative curve DSC2 in accordance with some embodiments of the present disclosure. FIG. 5 is a schematic diagram of an atypical growth type curve GC3 in accordance with some embodiments of the present disclosure. FIG. 6 is a schematic diagram of an atypical growth type curve GC4 in accordance with some embodiments of the present disclosure.

In some embodiments, the data of the typical growth type curve GC1 and the atypical growth type curves GC2-GC4 are stored in the memory 110, so that the processor 120 can obtain the data of the typical growth type curve GC1 and the atypical growth type curves GC2-GC4 according to the requirement. As shown in FIG. 3, the growth type curve GC1 has a plurality of data points, and each data point of the growth type curve GC1 is corresponding to one fluorescence intensity (i.e., “F” at a left vertical axis of FIG. 3) and one cycle number (i.e., “cycle” at a horizontal axis of FIG. 3). As shown in FIG. 4, the growth type curve GC2 also has a plurality of data points, and each data point of the growth type curve GC2 is also corresponding to one fluorescence intensity (i.e., “F” at a left vertical axis of FIG. 4) and one cycle number (i.e., “cycle” at a horizontal axis of FIG. 4). For simplifying description, the growth type curve GC3 of FIG. 5 and the growth type curve GC4 of FIG. 6 show part information only.

During the operation, the processor 120 first determines whether the growth type curve is typical or atypical. As shown in FIG. 3, the typical growth type curve GC1 is a S-shaped curve. The S-shaped curve is usually characterized by “inflection point”, that is, the slope before the inflection point is increasing gradually, and the slope after the inflection point is decreasing gradually. In other words, the inflection point is usually a point at which the curve has a maximum slope. Accordingly, the inflection point can be found efficiently according to the characteristic of the slope. On the other hand, the atypical growth type curve, for example, the growth type curve GC2 of FIG. 4, may not be the S-shaped curve. Therefore, it is difficult to find the characteristic of “inflection point” from the atypical growth type curve GC2. The processing approach for the atypical growth type curve GC2 will be described in detail later.

In operation S201, the processor 120 calculates a plurality of first derivative values (i.e., a plurality of slope values of the growth type curve GC1) corresponding to the data points of the growth type curve GC1, to form a first derivative curve DFC1 as shown in FIG. 3 (a right vertical axis of FIG. 3 represents the scale of the first derivative (VF)). Similarly, the processor 120 calculates a plurality of first derivative values (i.e., a plurality of slope values of the growth type curve GC2) corresponding to the data points of the growth type curve GC2, to form a first derivative curve DFC2 as shown in FIG. 4 (a right vertical axis of FIG. 4 represents the scale of the first derivative (VF)). In some embodiments, the data of the first derivative curve DFC1 and the first derivative curve DFC2 can be stored in the memory 110 to provide for the processor 120 to access.

In some embodiments, if the dispersion of the data points of the growth type curve is too high, the processor 120 can perform a smoothing process on the data points of the growth type curve before executing operation S201.

In operation S202, the processor 120 searches at least one local maximum value (i.e., finds several points that might be “inflection point”) from the first derivative values of the growth type curve GC1. It can be appreciated that the local maximum value of the first derivative values is usually a point at which the slope is turned from positive into negative. Therefore, the local maximum value of the first derivative values can be found efficiently according to the slope characteristic of the first derivative curve DFC1.

For searching the at least one local maximum value, the processor 120 calculates a plurality of second derivative values corresponding to the data points of the growth type curve GC1 first, to form a second derivative curve DSC1 as shown in FIG. 3 (the right vertical axis of FIG. 3 represents the scale of the first derivative (∇²F), the first derivative and the second derivative share the same scale basically). Each second derivative value represents the slope of a corresponding point on the first derivative curve DFC1. Then, the local maximum value of the first derivative values could be found by searching for the second derivative value that is between at least one positive value (corresponding to positive slope) and at least one negative value (corresponding to negative slope).

In some embodiments, the second derivative value that is between at least one positive value and at least one negative value could be found by multiplying two second derivative values corresponding to two adjacent cycle numbers. As shown in FIG. 3, the second derivative value corresponding to the cycle number “29” and the second derivative value corresponding to the cycle number “30” are multiplied to get the positive numeral value. The second derivative value corresponding to the cycle number “30” and the second derivative value corresponding to the cycle number “31” are multiplied to get the negative numeral value. The second derivative value corresponding to the cycle number “31” and the second derivative value corresponding to the cycle number “32” are multiplied to get the positive numeral value. Accordingly, the second derivative value that is between at least one positive value and at least one negative value might be corresponding to the cycle number “30” or “31”. Then, the processor 120 compares two derivative values corresponding to two cycle numbers “30” and “31”, and determines the greater one of two derivative values corresponding to two cycle numbers “30” and “31” as the local maximum value of the first derivative values. In the present embodiment, the processor 120 determines the first derivative value corresponding to the cycle number “30” on the first derivative curve DFC1 as a local maximum value Max1.

It can be appreciated that the first derivative values might include multiple local maximum values, but not all local maximum values are meaningful data. For example, the first derivative value corresponding to the cycle number “7” (or “40”) might be considered as the local maximum value. However, the first derivative value corresponding to the cycle number “7” (or “40”) is higher than several first derivative values adjacent thereto, possibly because of the jitter of the fluorescent signals. Accordingly, after finding the at least one local maximum value, the processor 120 would further determine whether the at least one local maximum value is meaningful data (i.e., whether has the characteristic of inflection point). It can be appreciated that the slopes near the inflection point on the S-shaped curve are all positive, and the first derivative value corresponding to the inflection point is the most significant. Therefore, it can be determined whether the at least one local maximum value is corresponding to the inflection point of the growth type curve according to characteristic of the slopes near the inflection point.

In operation S203, the processor 120 determines whether the at least one local maximum value is greater than a first threshold (e.g., 0.1), and determines whether a part of the first derivative values adjacent to the at least one local maximum value are all positive. In the example of the local maximum value Max1 as shown in FIG. 3, the local maximum value Max1 is corresponding to the cycle number “30”. The processor 120 would determine whether the local maximum value Max1is greater than 0.1 and determine whether the first derivative values corresponding to the adjacent cycle numbers “27”, “28”, “29”, “31”, “32” and “33” are all positive. As shown in FIG. 3, since the local maximum value Max1 is greater than 0.1 and the part of the first derivative values adjacent to the local maximum value Max1 are all positive, the data point corresponding to the cycle number “30” on the growth type curve GC1 is most likely to be “inflection point”. Accordingly, the processor 120 executes operation S204. In operation S204, the processor 120 determines the local maximum value Max1 as a target maximum value Maxt.

As described above, the growth type curve GC2 of FIG. 4 is not S-shaped curve and is likely to not have the significant characteristic of “inflection point”. Referring to FIG. 2A again, in operation S202, the processor 120 may not be able to find the local maximum value from the first derivative values of the growth type curve GC2 so as to execute operation S205. The descriptions of searching the local maximum value of the first derivative curve DFC2 is similar to those of the growth type curve GC1 (for example, calculating a plurality of second derivative values corresponding to the data points of the growth type curve GC2 to form the second derivative curve DSC2 as shown in FIG. 4), and therefore are omitted herein.

It can be appreciated that the atypical growth type curve GC2 does not represent that the target object (e.g., pathogen) is not present in the object under test, it may represent that the content of the target object is low. Because the target object with low content needs to be amplified more times, the test result of the target object may be reflected in several data points corresponding to the later cycle numbers on the growth type curve. For example, the slopes of the later data points may increase each by each. Accordingly, the processor 120 would determine whether the several data points corresponding to the later cycle numbers on the atypical growth type curve are meaningful data according to characteristic of the slopes (e.g., execute operation S205).

In operation S205, the processor 120 searches an incremental part of the first derivative values after a predetermined effective cycle number Cpd (for example, can but not limited to be the cycle number “35”). In practical, a portion after the predetermined effective cycle number Cpd on the growth type curve GC2 is known as gray zone. In the embodiment of FIG. 4, the processor 120 finds that several first derivative values corresponding to the cycle numbers “36” to “41” increase each by each (i.e., incremental), and therefore determines that the data points corresponding to the cycle numbers “36” to “41” may be meaningful data (that is, the target object may be present in the object under test, but the content thereof is low). After finding the incremental part of the first derivative values, the processor 120 executes operation S206. In operation S206, the processor 120 determines whether a last one of the incremental part of the first derivative values is greater than a second threshold (e.g., 0.03) to further check the effectiveness of data.

However, the present disclosure is not limited herein. In other embodiments, when the processor 120 finds that at least three first derivative values after the predetermined effective cycle number Cpd increase each by each and determines that the lase one of the at least three incremental first derivative values is greater than the second threshold, the processor 120 determines that the data points corresponding the at least three first derivative values are meaningful data.

In other embodiments, the processor 120 cannot find the incremental part of the first derivative values after the predetermined effective cycle number in operation S205 (which means that the target object may not exist), or determines that the last one of the incremental part of the first derivative values is not greater than the second threshold in operation S206. Accordingly, the processor 120 stops to execute the data sifting method 200.

After determining that the growth type curve GC2 is atypical curve and has meaningful data, the processor 120 executes operation S207. In operation S207, the processor determines the last one of the incremental part of the first derivative values as the target maximum value. In the embodiment of FIG. 4, the processor 120 determines the first derivative value corresponding to the cycle number “41” as the target maximum value Maxt.

After determining the target maximum value Maxt, the processor 120 executes operations S208-S209. In operation S208, the processor 120 obtains a target cycle number corresponding to the target maximum value Maxt (e.g., an inflection point cycle number C_{1/2} in FIG. 3, a gray zone cycle number Cg in FIG. 4). In the embodiment of FIG. 3, the inflection point cycle number C_{1/2} is the cycle number “30”. In the embodiment of FIG. 4, the gray zone cycle number Cg is the cycle number “41”.

In operation S209, the processor 120 derives a basic cycle number Cb according to the first derivative value corresponding to the target cycle number by counting backwards from the target cycle number with a predetermined number of cycle numbers. Specifically, the greater the first derivative value corresponding to the target cycle number, the less the number of counting. The smaller the first derivative value corresponding to the target cycle number, the more the number of counting. In the embodiment of FIG. 3, the processor 120 counts backwards from the inflection point cycle number C_{1/2} with seven cycle numbers according to the first derivative value corresponding to the inflection point cycle number C_{1/2}, so as to obtain the basic cycle number Cb (i.e., the cycle number “23”). In the embodiment of FIG. 4, the processor 120 counts backwards from the gray zone cycle number Cg with ten cycle numbers according to the first derivative value corresponding to the gray zone cycle number Cg, so as to obtain the basic cycle number Cb (i.e., the cycle number “31”).

After operation S209 is executed, in view of that some atypical growth type curve (e.g., GC3 in FIG. 5, GC4 in FIG. 6) different to the growth type curve GC2 are still present in practical, the processor 120 executed operation S210 to further verify the effectiveness of the target cycle number and the basic cycle number.

As shown in FIG. 5, since a steep ascent is present between the data points D15-D17 corresponding to the cycle numbers “15”-“17” on the growth type curve GC3 on which the smoothing process is not performed, the processor 120 may mistakenly determine the cycle number “16” as the target cycle number after executing the aforementioned operations on the growth type curve GC3. As shown in FIG. 6, it is assumed that the processor 120 executes the aforementioned operations on the growth type curve GC4 and derives that the basic cycle number is the cycle number “22”. Since a decline is present between the data points corresponding to the cycle numbers “1”-“25” on the growth type curve GC4, the processor 120 may be affected when executing the corresponding operations (e.g., operation S211) later according to the basic cycle number Cb.

As shown in FIG. 2B, in operation S210, the processor 120 checks whether the growth type curve on which the smoothing process is not performed has an abnormal curve characteristic according to the target cycle number (e.g., the inflection point cycle number C_{1/2} in FIG. 3, the gray zone cycle number Cg in FIG. 4, the cycle number “16” in FIG. 5) or/and the basic cycle number (e.g., the basic cycle number Cb in FIGS. 3 and 4, the cycle number “22” in FIG. 6).

In the embodiment of FIG. 5, the processor 120 subtracts the fluorescence intensity of each of the data points D15-D17 around the cycle number “16” (i.e., the target cycle number) by the fluorescence intensity of its previous data point (for example, the fluorescence intensity of the data point “16” is subtracted from the fluorescence intensity of the data point “17”, and the fluorescence intensity of the data point “15” is subtracted from the fluorescence intensity of the data point “16”), and calculates that a fluorescence intensity change (for example, divides a fluorescence intensity difference between the data points D15-D16 by the fluorescence intensity of the data point D15) is greater than a third threshold (e.g., can but not limited to be 50%), so as to determine that the growth type curve GC3 has the abnormal curve characteristic (i.e., the steep ascent). Accordingly, the processor 120 ends the data sifting method 200.

In the embodiment of FIG. 6, the processor 120 calculates the first derivative values of the data points (e.g., the data points corresponding to the cycle numbers “19”-“25”) around the cycle number “22” (i.e., the basic cycle number), and determines that the first derivative values of the data points around the cycle number “22” are all negative, so as to determine that the growth type curve GC4 has the abnormal curve characteristic (i.e., the decline).

In the embodiments of FIGS. 3-4, the processor 120 subtracts the fluorescence intensity of each of the data points around the target cycle number (i.e., the inflection point cycle number C_{1/2}, the gray zone cycle number Cg) by the fluorescence intensity of its previous data point and calculates that the fluorescence intensity change is not greater than the third threshold. In addition, the processor 120 calculates the first derivative values of the data points around the basic cycle number Cb and determines that the first derivative values of the data points around the basic cycle number Cb are not all negative. Accordingly, the processor 120 determines that the growth type curves GC1 and GC2 have no abnormal curve characteristic and executes operation S211 then.

In operation S211, the processor 120 adjusts the data points of the growth type curve according to the basic cycle number Cb to form a baseline (not shown) of the growth type curve. In the embodiment of FIG. 3, the data point corresponding to the basic cycle number Cb on the growth type curve GC1 is corresponding to the fluorescence intensity “0.2”. The processor 120 can regard the fluorescence intensity “0.2” as a basic data value and sets the data points corresponding to all cycle numbers before the basic cycle number Cb to correspond to the basic data value, to form the baseline. For example, the data point corresponding to the cycle number “15” on the growth type curve GC1 is corresponding to the fluorescence intensity “0.19” originally. After being set by the processor 120, the data point corresponding to the cycle number “15” would be corresponding to the fluorescence intensity “0.2” (i.e., the basic data value). The setting of the data points corresponding to other cycle numbers (e.g., “1”-“14” and “16”-“22”) is similar to those of the cycle number “15”, and therefor is omitted herein. In other embodiments, after executing operation S209, the processor 120 directly executes operation S211.

After the baseline of the growth type curve (GC1 or GC2) is set, a first Cq value is calculated according to the adjusted growth type curve (i.e., operation S212). In operation S212, the processor 120 performs a normalization operation and a calculation of fitting function (for example, fitting with Levenberg Marquardt method). Later, the processor 120 derives the first Cq value by the fitting function. In some embodiments, after executing operation S212, the processor directly executes operation S214.

In the embodiment of FIG. 2B, the processor 120 further executes operation S213 to verify the effectiveness of the first Cq value. In operation S213, the processor 120 increases or decreases the first threshold (in operation S203) or the second threshold (in operation S206) to recalculate at least one second Cq value. Specifically, the processor 120 can multiply or divide the first threshold (or the second threshold) by a coefficient (e.g., positive integer). In operation S213, the processor 120 further determines whether a difference value between the first Cq value and the at least one second Cq value is smaller than a predetermined value (e.g., 5%). For example, the first Cq value is 23.5, and the second Cq value recalculated by the processor 120 is 23.52. In such way, the difference value is 2%. If the difference value between the first Cq value and the at least one second Cq value is smaller than the predetermined value, it means that the calculation of the first Cq value is effective, so that the processor 120 executes operation S214.

In operation S214, the processor 120 interprets the test result of the object under test is positive (i.e., the target object is present in the object under test) or negative (i.e., the target object is not present in the object under test) according to the first Cq value, a difference value between the first cycle number and the last cycle number and coefficients of the fitting function.

In another example, the first Cq value is 23.5, and two second Cq values recalculated by the processor 120 are 24.32 and 22.7. In such way, two difference values corresponding to the two second Cq values are all not smaller than the predetermined value. If the difference value between the first Cq value and the at least one second Cq value is not smaller than the predetermined value, it means that the calculation of the first Cq value is invalid, so that the processor 120 executes operation S215.

In operation S215, the processor 120 chooses the smallest one from the first Cq value and the two recalculated second Cq values. In the above-described embodiment, the smallest one is one of the second Cq value (i.e., 22.7). The processor 120 then interprets the test result of the object under test is positive or negative according to the smallest one of the first Cq value and the at least one second Cq value, a difference value between the first cycle number and the last cycle number and coefficients of the fitting function. It can be appreciated that the number of recalculated second Cq value can be determined according to the requirement.

Referring to FIG. 2A again, in other embodiments, the processor 120 finds at least one local maximum value on the first derivative curve in operation S202, but determines that the part of the first derivative values adjacent to the at least one local maximum value on the first derivative curve are not all positive (or determines that the at least one local maximum value is not greater than the first threshold) in operation S203, so as to execute operation S205. The descriptions of other operations are similar to those of the above-described embodiments, and therefore are omitted herein.

In other embodiments, the fluorescent signals are unstable at first few cycle numbers due to the physical limitation (e.g., melted wax is present on test reagent). Accordingly, the processor 120 can limit a search range to be after a reference cycle number (e.g., the cycle number “10”) when searching the at least one local maximum value on the first derivative curve, so as to prevent the detected fluorescent signals from the influence of melted wax.

In sum, the data sifting apparatus 100 and the data sifting method 200 of the present disclosure find the meaningful data according to the characteristic of typical or atypical growth type curve (for example, the inflection point or the incremental part of the first derivative values at later cycle numbers) before calculating the Cq value, so as to reduce the calculation deviation due to the unstable fluorescent signals. In addition, after the Cq value is calculated, the present disclosure can recalculate the Cq value by adjusting the first threshold or the second threshold, so as to verify the effectiveness of the Cq value calculated previously. In such way, the condition that the interpretation of nucleic acid testing shows false positive or false negative is decreased.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the invention. In view of the foregoing, it is intended that the present invention cover modifications and variations of this invention provided they fall within the scope of the following claims.

What is claimed is:

1. A data sifting method applied to a processor, wherein the processor is coupled to a memory storing a growth type curve, the growth type curve is generated by performing a polymerase chain reaction on an object under test, the growth type curve comprises a plurality of data points, and the data sifting method comprises steps of:
calculating a plurality of first derivative values corresponding to the data points;
searching at least one local maximum value from the first derivative values;
determining whether the at least one local maximum value is greater than a first threshold, and determining whether a part of the first derivative values adjacent to the at least one local maximum value are all positive;
determining one of the first derivative values after a predetermined effective cycle number or the at least one local maximum value as a target maximum value according to a determination result;
obtaining a target cycle number corresponding to the target maximum value;
deriving a basic cycle number according to the first derivative value corresponding to the target cycle number;
adjusting the data points according to the basic cycle number to form a baseline of the growth type curve; and
calculating a first Cq value according to the adjusted growth type curve.

2. The data sifting method of claim 1, wherein searching the at least one local maximum value comprises steps of:
calculating a plurality of second derivative values corresponding to the data points;
multiplying two of the second derivative values corresponding to two adjacent cycle numbers; and
comparing two of the first derivative values corresponding to the two adjacent cycle numbers which have a negative multiplication result, and determining the larger one of the two of the first derivative values corresponding to the two adjacent cycle numbers as the at least one local maximum value.

3. The data sifting method of claim 1, wherein before adjusting the data points to form the baseline, the data sifting method further comprises a step of:
checking whether the growth type curve has an abnormal curve characteristic according to the target cycle number or the basic cycle number.

4. The data sifting method of claim 1, wherein determining the target maximum value comprises a step of:
when the at least one local maximum value is greater than the first threshold and the part of the first derivative values adjacent to the at least one local maximum value are all positive, determining the at least one local maximum value as the target maximum value.

5. The data sifting method of claim 1, wherein determining the target maximum value comprises s step of:
when the at least one local maximum value is not greater than the first threshold or the part of the first derivative values adjacent to the at least one local maximum value are not all positive, determining the one of the first derivative values after the predetermined effective cycle number as the target maximum value.

6. The data sifting method of claim 5, wherein determining the one of the first derivative values after the predetermined effective cycle number as the target maximum value comprises steps of:
searching an incremental part of the first derivative values after the predetermined effective cycle number; and
if a last one of the incremental part of the first derivative values is greater than a second threshold, determining the last one of the incremental part of the first derivative values as the target maximum value.

7. The data sifting method of claim 6, wherein after calculating the first Cq value, the data sifting method further comprises steps of:
adjusting the first threshold or the second threshold to recalculate at least one second Cq value;
if a difference value between the first Cq value and the at least one second Cq value is smaller than a predetermined value, interpreting a test result of the object under test is positive or negative according to the first Cq value; and
if the difference value between the first Cq value and the at least one second Cq value is not smaller than the predetermined value, interpreting the test result of the object under test is positive or negative according to the smallest one of the first Cq value and the at least one second Cq value.

8. The data sifting method of claim 1, wherein the data point corresponding to the basic cycle number has a basic data value, and adjusting the data points comprises a step of:
setting the data points corresponding to the cycle numbers before the basic cycle number to correspond to the basic data value, to form the baseline.

9. The data sifting method of claim 1, wherein when the at least one local maximum value is not found, the data sifting method further comprises steps of:
searching an incremental part of the first derivative values after the predetermined effective cycle number; and
if a last one of the incremental part of the first derivative values is greater than a second threshold, determining the last one of the incremental part of the first derivative values as the target maximum value.

10. The data sifting method of claim 9, wherein after calculating the first Cq value, the data sifting method further comprises steps of:
adjusting the first threshold or the second threshold to recalculate at least one second Cq value;
if a difference value between the first Cq value and the at least one second Cq value is smaller than a predetermined value, interpreting a test result of the object under test is positive or negative according to the first Cq value; and
if the difference value between the first Cq value and the at least one second Cq value is not smaller than the predetermined value, interpreting the test result of the object under test is positive or negative according to the smallest one of the first Cq value and the at least one second Cq value.

11. A data sifting apparatus, comprising:
a memory configured to store a growth type curve including a plurality of data points, wherein the growth type curve is generated by performing a polymerase chain reaction on an object under test; and
a processor coupled to the memory and configured to execute following operations:
calculating a plurality of first derivative values corresponding to the data points;
searching at least one local maximum value from the first derivative values;
determining whether the at least one local maximum value is greater than a first threshold, and determining whether a part of the first derivative values adjacent to the at least one local maximum value are all positive;
determining one of the first derivative values after a predetermined effective cycle number or the at least one local maximum value as a target maximum value according to a determination result;
obtaining a target cycle number corresponding to the target maximum value;
deriving a basic cycle number according to the first derivative value corresponding to the target cycle number;
adjusting the data points according to the basic cycle number to form a baseline of the growth type curve; and
calculating a first Cq value according to the adjusted growth type curve.

12. The data sifting apparatus of claim 11, wherein searching the at least one local maximum value comprises steps of:
calculating a plurality of second derivative values corresponding to the data points;
multiplying two of the second derivative values corresponding to two adjacent cycle numbers; and
comparing two of the first derivative values corresponding to the two adjacent cycle numbers which have a negative multiplication result, and determining the larger one of the two of the first derivative values corresponding to the two adjacent cycle numbers as the at least one local maximum value.

13. The data sifting apparatus of claim 11, wherein before adjusting the data points to form the baseline, the operations further comprise a step of:
checking whether the growth type curve has an abnormal curve characteristic according to the target cycle number or the basic cycle number.

14. The data sifting apparatus of claim 11, wherein determining the target maximum value comprises a step of:
when the at least one local maximum value is greater than the first threshold and the part of the first derivative values adjacent to the at least one local maximum value are all positive, determining the at least one local maximum value as the target maximum value.

15. The data sifting apparatus of claim 11, wherein determining the target maximum value comprises a step of:
when the at least one local maximum value is not greater than the first threshold or the part of the first derivative values adjacent to the at least one local maximum value are not all positive, determining the one of the first derivative values after the predetermined effective cycle number as the target maximum value.

16. The data sifting apparatus of claim 15, wherein determining the one of the first derivative values after the predetermined effective cycle number as the target maximum value comprises steps of:
searching an incremental part of the first derivative values after the predetermined effective cycle number; and
if a last one of the incremental part of the first derivative values is greater than a second threshold, determining the last one of the incremental part of the first derivative values as the target maximum value.

17. The data sifting apparatus of claim 16, wherein after calculating the first Cq value, the operations further comprise steps of:
adjusting the first threshold or the second threshold to recalculate at least one second Cq value;
if a difference value between the first Cq value and the at least one second Cq value is smaller than a predetermined value, interpreting a test result of the object under test is positive or negative according to the first Cq value; and
if the difference value between the first Cq value and the at least one second Cq value is not smaller than the predetermined value, interpreting the test result of the object under test is positive or negative according to the smallest one of the first Cq value and the at least one second Cq value.

18. The data sifting apparatus of claim 11, wherein the data point corresponding to the basic cycle number has a basic data value, and adjusting the data points comprises a step of:
setting the data points corresponding to the cycle numbers before the basic cycle number to correspond to the basic data value, to form the baseline.

19. The data sifting apparatus of claim 11, wherein when the at least one local maximum value is not found, the operations further comprise steps of:
searching an incremental part of the first derivative values after the predetermined effective cycle number; and
if a last one of the incremental part of the first derivative values is greater than a second threshold, determining the last one of the incremental part of the first derivative values as the target maximum value.

20. The data sifting apparatus of claim 19, wherein after calculating the first Cq value, the operations further comprise steps of:
adjusting the first threshold or the second threshold to recalculate at least one second Cq value;
if a difference value between the first Cq value and the at least one second Cq value is smaller than a predetermined value, interpreting a test result of the object under test is positive or negative according to the first Cq value; and
if the difference value between the first Cq value and the at least one second Cq value is not smaller than the predetermined value, interpreting the test result of the object under test is positive or negative according to the smallest one of the first Cq value and the at least one second Cq value.
